(19) United States
(12) Patent Application Publication
(10) Pub. No.: US 2026/0360544 A1
(43) Pub. Date: Nov. 12, 2026

(54) DIAGNOSIS EVALUATION AND SUBSTANTIATION USING GENERATIVE LANGUAGE MODELS

(71) Applicant: Ambience Healthcare, Inc., San Francisco, CA (US)

(72) Inventors: Patrick van Nieuwenhuizen, San Francisco, CA (US); Vinesh Girish Kannan, San Francisco, CA (US); Joshua James Cowdy, Madison, WI (US); Madison Enright, San Francisco, CA (US); Tanha Kabir, Seattle, WA (US); Alexander Flis Ryan, New York, NY (US); Arnelle Ansong Chang, San Francisco, CA (US)

(21) Appl. No.: 19/670,982

(22) Filed: May 7, 2026

Related U.S. Application Data
(60) Provisional application No. 63/801,443, filed on May 7, 2025.

(51) Int. Cl. G16H 50/20; G16H 80/00

(52) U.S. Cl. CPC G16H 50/20; G16H 80/00

(57) ABSTRACT

A physician documentation system groups a patient's diagnoses into clusters based on likelihoods of comorbidity, aggregates substantiation data for each cluster into a substantiation data set, and transmits an evaluation prompt for each cluster to a generative language model. The generative language model generates a measure of sufficiency for each cluster, and the physician documentation system updates a user interface element for each diagnosis based on those measures. In some embodiments, the physician documentation system detects a trigger event at a physician client application, generates an evaluation prompt for each diagnosis, identifies a sufficiency category for each measure of sufficiency, and updates a user interface element for each diagnosis based on the corresponding sufficiency category. In some embodiments, the physician documentation system generates a substantiation prompt for each diagnosis that instructs a generative language model to generate substantiation data based on the patient data and initial substantiation data.

### CROSS-REFERENCE TO RELATED APPLICATIONS

This application claims the benefit of U.S. Provisional Application No. 63/801,443, filed on May 7, 2025, which is hereby incorporated by reference in its entirety.

### BACKGROUND

In modern healthcare, accurate and comprehensive clinical documentation is critical for ensuring high-quality patient care and continuity of treatment. One of the primary forms of such documentation is physician notes, which are notes written by a physician or their assistant that serve as records of a patient's appointment. These notes typically include patient history, examination findings, assessments, diagnoses, and treatment plans. Beyond guiding immediate clinical decisions, physician notes form the foundation for communication among healthcare providers, facilitate long-term care management, and serve as key evidence in regulatory audits and legal matters.

A crucial aspect of clinical documentation, particularly in relation to diagnosis coding and risk adjustment, is compliance with established documentation standards such as the MEAT criteria—Monitor, Evaluate, Assess, and Treat. The MEAT framework ensures that diagnoses recorded in patient charts are not only listed but are actively addressed and substantiated within the medical record. Proper adherence to standards like MEAT is essential for validating the medical necessity of services rendered and for supporting accurate coding.

However, despite its importance, clinical documentation can include errors, omissions, and inconsistencies. For example, a physician’s notes may be incomplete or inaccurate due to time constraints, administrative burdens, and reliance on memory during or after patient encounters. Additionally, the increasing use of electronic systems for maintaining health records can sometimes encourage templated or "copy-paste" behaviors, resulting in generic or outdated information that fails to reflect the specific details of a patient's condition or treatment.

The consequences of inadequate documentation are significant. For example, poor documentation can lead to gaps in patient care, miscommunication between providers, and potential medical errors. These problems can be difficult to correct once detected, as a significant amount of time may have passed since the records were created and the physician’s memory of the session may have degraded.

Computing systems are generally unable to automatically detect and address these documentation inadequacies. Physician notes are generally unstructured data, meaning that the information contained in those notes is expressed in free-form natural language rather than in a predefined, machine-readable format. Computing systems typically require structured data, such as coded fields or standardized entries, to perform automated processing and analysis. For example, a computing system may readily process a structured data field indicating that a patient's blood pressure reading is 140/90 mmHg, but may be unable to extract the same information from a physician's free-form note stating that the patient presented with elevated blood pressure during the visit. Because physician notes do not conform to a fixed structure, computing systems cannot reliably parse, interpret, or evaluate the content of those notes using conventional data processing techniques.

### SUMMARY

A physician documentation system groups a patient's diagnoses into clusters based on likelihoods of comorbidity and uses a generative language model to evaluate whether the substantiation data for each cluster is sufficient. The physician documentation system accesses patient data and receives patient diagnosis data that includes a set of diagnoses and substantiation data for each of those diagnoses. The physician documentation system generates diagnosis clusters by grouping together diagnoses that have likelihoods of comorbidity exceeding a threshold value, and aggregates the substantiation data for the diagnoses in each cluster into a substantiation data set. The physician documentation system generates an evaluation prompt for each diagnosis cluster that includes the diagnoses, the substantiation data set, and instructions for a generative language model to generate a measure of sufficiency of the substantiation data for those diagnoses. The physician documentation system transmits the evaluation prompts to the generative language model, receives a measure of sufficiency for each diagnosis cluster in response, and updates a user interface element for each of the diagnoses based on those measures of sufficiency.

A physician documentation system monitors for trigger events at a physician client application and, in response to each trigger event, evaluates the substantiation data for each of a patient's diagnoses and updates the user interface to reflect the sufficiency of that data. The physician documentation system detects a trigger event at a physician client application and, in response, accesses patient data and receives patient diagnosis data comprising a set of diagnoses and substantiation data for each of those diagnoses. The physician documentation system generates an evaluation prompt for each diagnosis that includes the diagnosis, the corresponding substantiation data, and instructions for a generative language model to generate a measure of sufficiency of the substantiation data for that diagnosis. The physician documentation system transmits the evaluation prompts to the generative language model and receives a measure of sufficiency for each diagnosis in response. The physician documentation system identifies a sufficiency category for each diagnosis from a set of sufficiency categories based on the corresponding measure of sufficiency, and updates a user interface element for each diagnosis in the user interface based on the corresponding sufficiency category.

A physician documentation system uses a generative language model to generate additional substantiation data for each of a patient's diagnoses based on the patient data and the initial substantiation data provided by the physician, and updates the user interface to include that additional substantiation data. The physician documentation system accesses patient data and receives patient diagnosis data comprising a set of diagnoses and initial substantiation data for each of those diagnoses, where the initial substantiation data is substantiation data initially generated by the physician for a diagnosis. The physician documentation system generates a substantiation prompt for each diagnosis that includes the diagnosis, the corresponding initial substantiation data, the patient data, and instructions for a generative language model to generate additional substantiation data for the diagnosis based on the initial substantiation data and the patient data. The physician documentation system transmits the substantiation prompts to the generative language model and receives additional substantiation data for each diagnosis in response. The physician documentation system updates a user interface element for each diagnosis to include the additional substantiation data.

### BRIEF DESCRIPTION OF THE DRAWINGS

FIG. 1 illustrates an example system environment for a physician documentation system, in accordance with one or more embodiments.

FIG. 2 is a flowchart for a method of clustering diagnoses for substantiation evaluation, in accordance with some embodiments.

FIG. 3 is a flowchart for a method of dynamically notifying a physician regarding the sufficiency in substantiation data, in accordance with some embodiments.

FIG. 4 is a flowchart for a method of generating additional substantiation data for patient diagnoses, in accordance with some embodiments.

FIG. 5 illustrates an example of how the physician documentation system generates diagnosis clusters from a set of diagnoses and aggregates substantiation data into substantiation data sets for each of those diagnosis clusters, in accordance with some embodiments.

FIG. 6 illustrates example diagnosis user interface elements displayed in the user interface of the physician client application, in accordance with some embodiments.

FIG. 7 illustrates additional substantiation data being displayed to a user through a user interface of a physician client application, in accordance with some embodiments.

### DETAILED DESCRIPTION

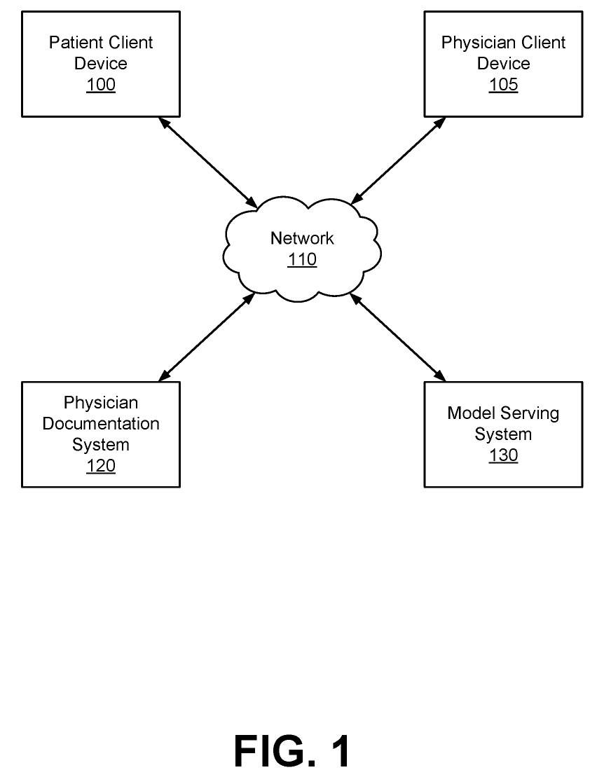

FIG. 1 illustrates an example system environment for a physician documentation system 120, in accordance with one or more embodiments. The system environment illustrated in FIG. 1 includes a patient client device 100, a physician client device 105, a network 110, a physician documentation system 120, and a model serving system 130. Alternative embodiments may include more, fewer, or different components from those illustrated in FIG. 1, and the functionality of each component may be divided between the components differently from the description below. Additionally, each component may perform their respective functionalities in response to a request from a human, or automatically without human intervention.

The patient client device 100 and the physician client device 105 are client devices through which a patient or physician may interact with the physician documentation system 120. The client device can be a personal or mobile computing device, such as a smartphone, a tablet, a laptop computer, or desktop computer. In some embodiments, the client devices execute a client application that uses an application programming interface (API) to communicate with the physician documentation system 120.

The patient client device 100 is a client device operated by a patient that allows the patient to access the physician documentation system 120. The patient may use the patient client device 100 to review patient data stored by the physician documentation system 120, such as health records, test results, and prior physician notes. The patient client device 100 may execute a client application that provides a user interface through which the patient may interact with the physician documentation system 120. For example, the patient may use the user interface to send messages to a physician, request appointments or prescription renewals, and submit health information such as symptom reports or responses to intake questionnaires. The patient client device 100 may also allow the patient to review summaries of prior sessions with a physician, including transcripts or summarized versions of those sessions generated by the physician documentation system 120. In some embodiments, the patient client device 100 may capture audio data of a session between the patient and a physician and transmit that audio data to the physician documentation system 120 for transcription and storage.

The physician client device 105 is a client device operated by a physician that allows the physician to access and interact with the physician documentation system 120. The physician client device 105 may execute a client application that provides a user interface through which the physician may generate and update patient health records. For example, the physician may use the user interface to input physician notes describing a patient's visit, including patient history, examination findings, assessments, and treatment plans. The physician client device 105 may also provide user interface elements through which the physician may input diagnoses for a patient. For example, the physician may select an ICD-10 or HCC code for a diagnosis, and the user interface may correspondingly update to provide an additional user interface element for inputting notes about that diagnosis. The physician may use the physician client device 105 to review test results, lab reports, and scan results stored by the physician documentation system 120, as well as to send messages to a patient. In some embodiments, the physician client device 105 may capture audio data of a session between the physician and a patient and may transmit that audio data to the physician documentation system 120 for transcription. The physician client device 105 may also allow the physician to review and verify substantiation data generated by the physician documentation system 120 for the physician's diagnoses before that data is saved to the patient's record.

The physician documentation system 120 is an online system that stores patient health records and supports physician documentation workflows. The physician documentation system 120 may store a variety of patient health records, such as physician notes, lab results, scan results, imaging reports, and medication histories. The physician documentation system 120 may allow a physician to generate and update these records through a client application or web browser operating on the physician client device 105. The physician documentation system 120 may also store transcripts of sessions between a patient and a physician, which the physician documentation system 120 may generate by processing audio data captured by the patient client device 100 or the physician client device 105. In some embodiments, the physician documentation system 120 may generate a summarized version of a transcript, for example by applying a generative language model to the transcript to produce a condensed summary of the session. The physician documentation system 120 may also monitor physician notes to evaluate whether those notes sufficiently substantiate the diagnoses that a physician has recorded for a patient, and may provide real-time feedback to the physician through the user interface of the physician client device 105. In some embodiments, the physician documentation system 120 may further generate additional substantiation data for a diagnosis based on stored patient data, and may present that additional substantiation data to the physician for review and verification before saving it to the patient's record.

The patient client device 100, the physician client device 105, the physician documentation system 120, and the model serving system 130 can communicate with each other via the network 110. The network 110 is a collection of computing devices that communicate via wired or wireless connections. The network 110 may include one or more local area networks (LANs) or one or more wide area networks (WANs). The network 110, as referred to herein, is an inclusive term that may refer to any or all of the standard layers used to describe a physical or virtual network, such as the physical layer, the data link layer, the network layer, the transport layer, the session layer, the presentation layer, and the application layer. The network 110 may include physical media for communicating data from one computing device to another computing device, such as multiprotocol label switching (MPLS) lines, fiber optic cables, cellular connections (e.g., 3G, 4G, or 5G spectra), or satellites. The network 110 also may use networking protocols, such as TCP/IP, HTTP, SSH, SMS, or FTP, to transmit data between computing devices. In some embodiments, the network 110 may include Bluetooth or near-field communication (NFC) technologies or protocols for local communications between computing devices. The network 110 may transmit encrypted or unencrypted data.

The model serving system 130 receives requests from the physician documentation system 120 to perform tasks using machine-learned models. The tasks include, but are not limited to, natural language processing (NLP) tasks, audio processing tasks, image processing tasks, video processing tasks, and the like. In one embodiment, the machine-learned models deployed by the model serving system 130 are models configured to perform one or more NLP tasks. The NLP tasks include, but are not limited to, text generation, query processing, machine translation, chatbots, and the like. In one embodiment, the language model is configured as a transformer neural network architecture. Specifically, the transformer model is coupled to receive sequential data tokenized into a sequence of input tokens and generates a sequence of output tokens depending on the task to be performed.

The model serving system 130 receives a request including input data (e.g., text data, audio data, image data, or video data) and encodes the input data into a set of input tokens. The model serving system 130 applies the machine-learned model to generate a set of output tokens. Each token in the set of input tokens or the set of output tokens may correspond to a text unit. For example, a token may correspond to a word, a punctuation symbol, a space, a phrase, a paragraph, and the like. For an example query processing task, the language model may receive a sequence of input tokens that represent a query and generate a sequence of output tokens that represent a response to the query. For a translation task, the transformer model may receive a sequence of input tokens that represent a paragraph in German and generate a sequence of output tokens that represents a translation of the paragraph or sentence in English. For a text generation task, the transformer model may receive a prompt and continue the conversation or expand on the given prompt in human-like text.

When the machine-learning model is a language model, the sequence of input tokens or output tokens are arranged as a tensor with one or more dimensions, for example, one dimension, two dimensions, or three dimensions. For example, one dimension of the tensor may represent the number of tokens (e.g., length of a sentence), one dimension of the tensor may represent a sample number in a batch of input data that is processed together, and one dimension of the tensor may represent a space in an embedding space. However, it is appreciated that in other embodiments, the input data or the output data may be configured as any number of appropriate dimensions depending on whether the data is in the form of image data, video data, audio data, and the like. For example, for three-dimensional image data, the input data may be a series of pixel values arranged along a first dimension and a second dimension, and further arranged along a third dimension corresponding to RGB channels of the pixels.

In one embodiment, the language models are large language models (LLMs) that are trained on a large corpus of training data to generate outputs for the NLP tasks. An LLM may be trained on massive amounts of text data, often involving billions of words or text units. The large amount of training data from various data sources allows the LLM to generate outputs for many tasks. An LLM may have a significant number of parameters in a deep neural network (e.g., transformer architecture), for example, at least 1 billion, at least 15 billion, at least 135 billion, at least 175 billion, at least 500 billion, at least 1 trillion, at least 1.5 trillion parameters.

Since an LLM has significant parameter size and the amount of computational power for inference or training the LLM is high, the LLM may be deployed on an infrastructure configured with, for example, supercomputers that provide enhanced computing capability (e.g., graphic processor units) for training or deploying deep neural network models. In one instance, the LLM may be trained and deployed or hosted on a cloud infrastructure service. The LLM may be pre-trained by the online system 120 or one or more entities different from the online system 120. An LLM may be trained on a large amount of data from various data sources. For example, the data sources include websites, articles, posts on the web, and the like. From this massive amount of data coupled with the computing power of LLM's, the LLM is able to perform various tasks and synthesize and formulate output responses based on information extracted from the training data.

In one embodiment, when the machine-learned model including the LLM is a transformer-based architecture, the transformer has a generative pre-training (GPT) architecture including a set of decoders that each perform one or more operations to input data to the respective decoder. A decoder may include an attention operation that generates keys, queries, and values from the input data to the decoder to generate an attention output. In another embodiment, the transformer architecture may have an encoder-decoder architecture and includes a set of encoders coupled to a set of decoders. An encoder or decoder may include one or more attention operations.

While a LLM with a transformer-based architecture is described as a primary embodiment, it is appreciated that in other embodiments, the language model can be configured as any other appropriate architecture including, but not limited to, long short-term memory (LSTM) networks, Markov networks, BART, generative-adversarial networks (GAN), diffusion models (e.g., Diffusion-LM), and the like.

In one embodiment, the task for the model serving system 130 is based on knowledge of the physician documentation system 120 that is fed to the machine-learned model of the model serving system 130, rather than relying on general knowledge encoded in the model weights of the model. Thus, one objective may be to perform various types of queries on the external data in order to perform any task that the machine-learned model of the model serving system 130 could perform. For example, the task may be to perform question-answering, text summarization, text generation, and the like based on information contained in an external dataset.

Thus, in one embodiment, the physician documentation system 120 is connected to an interface system. The interface system receives external data from the physician documentation system 120 and builds a structured index over the external data using, for example, another machine-learned language model or heuristics. The interface system receives one or more queries from the physician documentation system 120 on the external data. The interface system constructs one or more prompts for input to the model serving system 130. A prompt may include the query of the user and context obtained from the structured index of the external data. In one instance, the context in the prompt includes portions of the structured indices as contextual information for the query. The interface system obtains one or more responses from the model serving system and synthesizes a response to the query on the external data. While the physician documentation system 120 can generate a prompt using the external data as context, often times, the amount of information in the external data exceeds prompt size limitations configured by the machine-learned language model. The interface system can resolve prompt size limitations by generating a structured index of the data and offers data connectors to external data sources.

The example system environment in FIG. 1 illustrates an environment where the model serving system 130 is managed by a separate entity from the physician documentation system 120. However, in alternative embodiments, the model serving system 130 (or the interface system) is part of the physician documentation system 120.

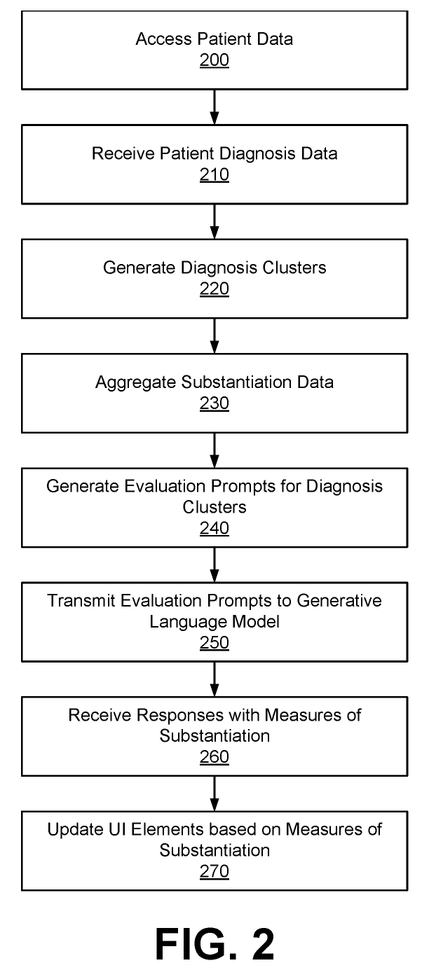

FIG. 2 is a flowchart for a method of clustering diagnoses for substantiation evaluation, in accordance with some embodiments. Alternative embodiments may include more, fewer, or different steps from those illustrated in FIG. 2, and the steps may be performed in a different order from that illustrated in FIG. 2. These steps may be performed by a physician documentation system (e.g., physician documentation system 120). Additionally, each of these steps may be performed automatically by the physician documentation system without human intervention.

The physician documentation system accesses 200 patient data for a patient. Patient data is data that describes health or medical characteristics of a patient. For example, the patient data may include test results, such as laboratory test results or imaging results. The patient data may also include physician notes generated by a physician for prior appointments with the patient. The patient data may further include medication histories, referral records, and prior diagnosis records associated with the patient. The physician documentation system may access patient data that is stored by the physician documentation system or by a third-party system in communication with the physician documentation system.

The patient data may also include a transcript of a session between the patient and a physician. The physician client device may record audio data of a session between the patient and the physician. The physician client device may generate text data of the transcript from the recorded audio data. For example, the physician client device may generate the transcript locally by applying a speech-to-text model to the audio data. Alternatively, the physician client device may transmit the audio data to the physician documentation system, and the physician documentation system may generate the transcript from the audio data. The transcript may be a direct transcript of the session. The transcript may also be a summarized version of the session. For example, the physician documentation system may apply a generative language model to the direct transcript to generate a summarized version of the transcript.

The physician documentation system receives 210 patient diagnosis data for the patient. Patient diagnosis data is data that describes diagnoses made by a physician. The patient diagnosis data may include a list of diagnoses of the patient, such as a list of International Classification of Diseases, Tenth Revision (ICD-10) codes or Hierarchical Condition Category (HCC) codes, as well as substantiation data for each of those diagnoses. The patient diagnosis data may also include substantiation data for each of the diagnoses. Substantiation data is patient data that substantiates a diagnosis. For example, the substantiation data for a diagnosis may include physician notes generated by the physician that describe the basis for the diagnosis, or health records that the physician has identified as relevant to the diagnosis, such as laboratory test results, imaging results, or session transcripts. The same patient data may be used to substantiate different diagnoses. For example, a laboratory test result showing elevated blood glucose levels may serve as substantiation data for both a diagnosis of type 2 diabetes mellitus and a diagnosis of diabetic peripheral neuropathy.

The physician may generate the patient diagnosis data through a user interface operating on the physician client device. For example, a physician may select diagnoses from a reference list of standardized codes, select portions of the patient data to associate with each diagnosis as substantiation data, or input notes for a diagnosis through a text input field. The physician documentation system may receive the patient diagnosis data from the physician client device and store the patient diagnosis data in association with the patient's health records.

The physician documentation system generates 220 a set of diagnosis clusters based on the patient diagnosis data. A diagnosis cluster is a cluster of diagnoses that are sufficiently related for the purposes of substantiation. For example, a diagnosis cluster may include diagnoses such as type 2 diabetes mellitus and diabetic peripheral neuropathy, which are closely related conditions where substantiation data for one diagnosis may also serve to substantiate the other. Similarly, a diagnosis cluster may include diagnoses such as hypertension and chronic kidney disease, or obesity and obstructive sleep apnea, where the clinical relationship between the diagnoses makes it appropriate to evaluate their substantiation data together.

The physician documentation system may generate diagnosis clusters based on likelihoods of comorbidity of the patient's diagnoses. For example, the physician documentation system may compute a likelihood of comorbidity for each pair of diagnoses in the patient diagnosis data and may use the likelihoods of comorbidity to generate the diagnosis clusters. The physician documentation system may compute the likelihood of comorbidity for a pair of diagnoses based on historical medical data for the diagnoses. For example, the physician documentation system may access a database of historical patient records and compute the frequency with which two diagnoses appear together across that population of patients. The physician documentation system may also compute the likelihood of comorbidity by applying a machine learning model to the pair of diagnoses. For example, the physician documentation system may apply a machine learning model that is trained on historical patient data to generate a likelihood of comorbidity score for each pair of diagnoses. The physician documentation system may also compute the likelihood of comorbidity based on patient data of other patients stored within the physician documentation system.

The physician documentation system identifies which diagnoses have a likelihood of comorbidity that exceeds a threshold likelihood of comorbidity. The physician documentation system may cluster together a pair of diagnoses when the likelihood of comorbidity for that pair exceeds the threshold likelihood of comorbidity. In some embodiments, the physician documentation system may generate a diagnosis cluster that includes exclusively diagnoses for which all pairwise likelihoods of comorbidity exceed the threshold likelihood of comorbidity. In other embodiments, the physician documentation system may add a diagnosis to an existing cluster when the likelihood of comorbidity between that diagnosis and at least one other diagnosis already in the cluster exceeds the threshold likelihood of comorbidity. The physician documentation system may apply either approach, or a combination of both approaches, when generating the set of diagnosis clusters.

The physician documentation system aggregates 230 the substantiation data for each of the diagnosis clusters to generate a substantiation data set for each diagnosis cluster. The physician documentation system may aggregate the substantiation data for a diagnosis cluster by identifying the substantiation data associated with each of the diagnoses in that cluster. The physician documentation system may then collect all of the identified substantiation data into a single aggregated substantiation data set for the diagnosis cluster. For example, if a diagnosis cluster includes three diagnoses, the physician documentation system may retrieve the substantiation data associated with each of those three diagnoses and combine that substantiation data into a single substantiation data set. In some embodiments, the physician documentation system may also deduplicate the substantiation data when generating the substantiation data set. For example, where the same patient data, such as a laboratory test result, has been identified as substantiation data for more than one diagnosis in the cluster, the physician documentation system may include that patient data only once in the substantiation data set. The physician documentation system may perform this aggregation process for each of the diagnosis clusters in the set of diagnosis clusters.

The physician documentation system generates 240 an evaluation prompt for each of the diagnosis clusters. An evaluation prompt is a prompt to a generative language model that instructs the generative language model to generate a measure of sufficiency of the substantiation data for the diagnoses in a diagnosis cluster. A measure of sufficiency is a value that indicates whether, or to what extent, the substantiation data is sufficient for the diagnoses in the diagnosis cluster. For example, the measure of sufficiency may be a binary value indicating whether the substantiation data is sufficient or insufficient for the diagnoses in the diagnosis cluster. The measure of sufficiency may also be a numerical score indicating a likelihood that the diagnoses in the cluster are accurate given the substantiation data. In some embodiments, the physician documentation system may generate a measure of sufficiency on a per-diagnosis basis, such that the generative language model generates a separate measure of sufficiency for each individual diagnosis in the diagnosis cluster. The physician documentation system may generate a measure of sufficiency for the diagnosis clusters based on the per-diagnosis measures of sufficiency.

An evaluation prompt for a diagnosis cluster may include a corresponding set of diagnoses for the diagnosis cluster and aggregated substantiation data for that cluster. The evaluation prompt may further include instructions to generate a measure of sufficiency of the substantiation data set for the subset of diagnoses. In some embodiments, the evaluation prompt may also include additional patient data beyond the substantiation data. For example, the physician documentation system may include the full patient data accessed for the patient, such as prior health records or session transcripts, to provide the generative language model with broader context for evaluating the substantiation data.

The evaluation prompt may also include diagnosis substantiation criteria for the diagnoses in the diagnosis cluster. Diagnosis substantiation criteria are criteria for whether substantiation data is sufficient for a diagnosis. For example, the diagnosis substantiation criteria may include the Monitor, Evaluate, Assess, and Treat (MEAT) criteria, which specify that a diagnosis should be monitored, evaluated, assessed, and treated within the patient's health records. The diagnosis substantiation criteria may also include diagnosis-specific criteria, such as a list of symptoms that are typically associated with a diagnosis, laboratory tests or imaging studies that should be conducted to confirm a diagnosis, or clinical guidelines published by third-party medical organizations such as the American Diabetes Association or the American Heart Association. The physician documentation system may store diagnosis substantiation criteria for each of a set of diagnoses in a database. The physician documentation system may retrieve the diagnosis substantiation criteria for each of the diagnoses in a diagnosis cluster and include those criteria in the evaluation prompt for that cluster.

The physician documentation system transmits 250 each of the evaluation prompts to a generative language model. The physician documentation system may transmit the evaluation prompts to the model serving system via the network. The physician documentation system may transmit the evaluation prompts sequentially, one for each diagnosis cluster, or may transmit multiple evaluation prompts in parallel. The physician documentation system may format each evaluation prompt as a text input that the generative language model is configured to receive and process.

The physician documentation system receives 260 a response from the generative language model for each of the evaluation prompts. Each response includes a measure of sufficiency for the corresponding diagnosis cluster. The physician documentation system may extract the measure of sufficiency from each response by parsing the text output generated by the generative language model. For example, the physician documentation system may parse the response to identify a numerical score, a binary value, or a category label that the generative language model has generated as the measure of sufficiency. The physician documentation system may store the extracted measures of sufficiency in association with the corresponding diagnosis clusters and the corresponding diagnoses.

The physician documentation system updates user interface elements 270 in the user interface of the physician client device based on the measures of sufficiency. The physician documentation system may update a user interface element for each of the diagnoses to indicate whether that diagnosis is sufficiently substantiated. For example, the physician documentation system may update the color of a user interface element associated with a diagnosis to reflect the measure of sufficiency for that diagnosis. The physician documentation system may display a user interface element in green when the substantiation data for a diagnosis is sufficient, in yellow when the substantiation data is partially sufficient, and in red when the substantiation data is insufficient. The physician documentation system may also update a user interface element to display a numerical score or a text label indicating the measure of sufficiency for a diagnosis. For example, the physician documentation system may display a label such as "Sufficient," "Needs Review," or "Insufficient" adjacent to a diagnosis in the user interface. In some embodiments, the physician documentation system may also update a user interface element to display a tooltip or an expandable panel that provides additional detail about why the substantiation data for a diagnosis received a particular measure of sufficiency. The physician documentation system may transmit instructions to the physician client device to update the user interface elements, and the physician client device may render the updated user interface elements in the client application or web browser.

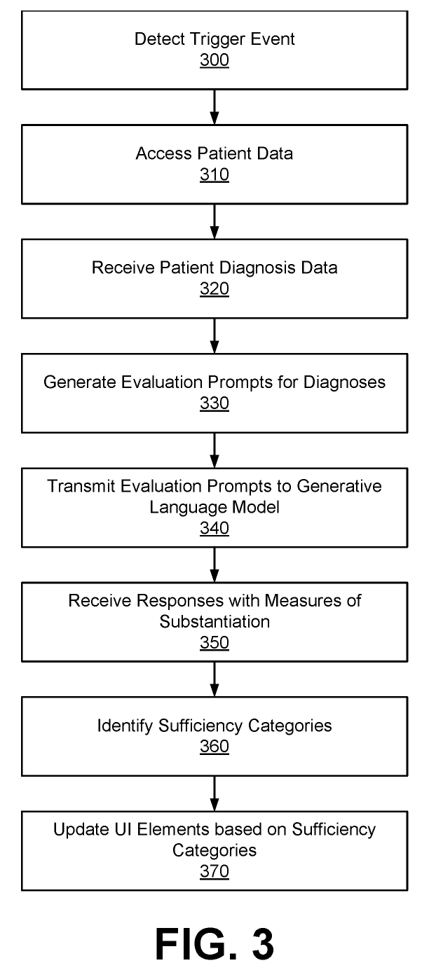

FIG. 3 is a flowchart for a method of dynamically notifying a physician regarding the sufficiency in substantiation data, in accordance with some embodiments. Alternative embodiments may include more, fewer, or different steps from those illustrated in FIG. 3, and the steps may be performed in a different order from that illustrated in FIG. 3. These steps may be performed by a physician documentation system (e.g., physician documentation system 120). Additionally, each of these steps may be performed automatically by the physician documentation system without human intervention.

The physician documentation system detects 300 a trigger event at a physician client application operating on the physician client device. A trigger event is an event that causes the physician documentation system to evaluate the substantiation data provided by a physician for a diagnosis. For example, a trigger event may occur when a physician adds new text to or modifies existing physician notes for a patient in the user interface. A trigger event may also occur when new patient data is received by the physician documentation system, such as when a new laboratory test result or imaging result becomes available for the patient. A trigger event may further occur when a physician adds a new diagnosis for a patient or modifies an existing diagnosis in the user interface. In some embodiments, a trigger event may occur when a physician selects a user interface element in the physician client application to manually request a sufficiency evaluation for one or more diagnoses. The physician documentation system may also detect a trigger event on a regular interval, such as every few minutes during an active session, so that the physician documentation system may periodically re-evaluate the substantiation data as the physician continues to generate and update records. Additional trigger events may include a physician signing or finalizing a patient record, a change in a patient's medication list, or the receipt of a referral or consultation note from another provider. The physician client application may monitor for these events and may transmit a notification to the physician documentation system when a trigger event occurs.

The physician documentation system accesses 310 patient data for the patient. The physician documentation system may access patient data stored by the physician documentation system, such as health records, test results, physician notes, and session transcripts. The physician documentation system may also access patient data stored by a third-party system in communication with the physician documentation system. For example, the physician documentation system may retrieve recently updated records, such as a new laboratory test result or an updated medication list, that have become available since the last evaluation was performed.

The physician documentation system receives 320 patient diagnosis data for the patient through the user interface operating on the physician client device. The patient diagnosis data may include a list of diagnoses of the patient and substantiation data for each of those diagnoses. For example, the patient diagnosis data may include ICD-10 or HCC codes selected by the physician, as well as physician notes and health records that the physician has identified as substantiation data for each diagnosis. The physician documentation system may retrieve the patient diagnosis data that is currently stored in association with the patient's health records, including any updates that the physician has made up to and including the trigger event.

The physician documentation system generates 330 an evaluation prompt for each of the diagnoses in the patient diagnosis data. Each evaluation prompt may include a corresponding diagnosis, the corresponding substantiation data for that diagnosis, and instructions to generate a measure of sufficiency of the substantiation data for that diagnosis. The evaluation prompt may also include additional patient data beyond the substantiation data, such as session transcripts or prior health records, to provide the generative language model with broader context for evaluating the substantiation data. The evaluation prompt may further include diagnosis substantiation criteria for the corresponding diagnosis, such as the MEAT criteria or diagnosis-specific clinical guidelines, as described above. In some embodiments, rather than generating an evaluation prompt for each diagnosis individually, the physician documentation system may generate evaluation prompts for diagnosis clusters, as described above with respect to FIG. 2.

The physician documentation system transmits 340 each of the evaluation prompts to a generative language model. The physician documentation system may transmit the evaluation prompts to the model serving system via the network. The physician documentation system may transmit the evaluation prompts sequentially, one for each diagnosis, or may transmit multiple evaluation prompts in parallel. The physician documentation system receives 350 a response from the generative language model for each of the evaluation prompts. Each response includes a measure of sufficiency for the corresponding diagnosis. The physician documentation system may extract the measure of sufficiency from each response by parsing the text output generated by the generative language model.

The physician documentation system identifies 360 a sufficiency category for each of the measures of sufficiency. A sufficiency category is a category from a set of sufficiency categories that represents a level or range of measures of sufficiency. For example, the set of sufficiency categories may include three categories, such as "Insufficient," "Partially Sufficient," and "Sufficient," where each category corresponds to a range of numerical scores generated by the generative language model. The physician documentation system may map a measure of sufficiency to a sufficiency category by comparing the measure of sufficiency to a set of threshold values that define the boundaries between categories. For example, the physician documentation system may assign a measure of sufficiency below a first threshold to the "Insufficient" category, a measure of sufficiency between the first threshold and a second threshold to the "Partially Sufficient" category, and a measure of sufficiency above the second threshold to the "Sufficient" category. In some embodiments, the set of sufficiency categories may include only two categories, such as "Sufficient" and "Insufficient," where the physician documentation system maps each measure of sufficiency to one of the two categories based on a single threshold value. In other embodiments, the set of sufficiency categories may include four or more categories to provide a more granular representation of the measure of sufficiency. The physician documentation system may also define sufficiency categories based on qualitative labels generated directly by the generative language model, where the generative language model selects a category label from a predefined set of candidate labels as part of its response. The physician documentation system identifies a sufficiency category for each of the diagnoses in the patient diagnosis data based on the corresponding measure of sufficiency.

The physician documentation system updates 370 the user interface element in the physician client application for each of the diagnoses based on the corresponding sufficiency category. The physician documentation system may update the user interface element for a diagnosis to indicate the sufficiency category for that diagnosis. For example, the physician documentation system may update the color of the user interface element based on the sufficiency category for the corresponding diagnosis, such as displaying the user interface element in red for a diagnosis in the "Insufficient" category, in yellow for a diagnosis in the "Partially Sufficient" category, and in green for a diagnosis in the "Sufficient" category. The physician documentation system may also update the user interface element to display a text label corresponding to the sufficiency category, such as "Insufficient," "Partially Sufficient," or "Sufficient," adjacent to the diagnosis in the user interface. In some embodiments, the physician documentation system may update the user interface element to display an icon that corresponds to the sufficiency category, such as a warning symbol for a diagnosis in the "Insufficient" category or a checkmark for a diagnosis in the "Sufficient" category. The physician documentation system may also update the user interface element to display an expandable panel that provides a summary of the substantiation evaluation for the corresponding diagnosis, including the measure of sufficiency and any diagnosis substantiation criteria that were not satisfied. The physician documentation system may transmit instructions to the physician client device to render the updated user interface elements, and the physician client device may update the display of the physician client application accordingly.

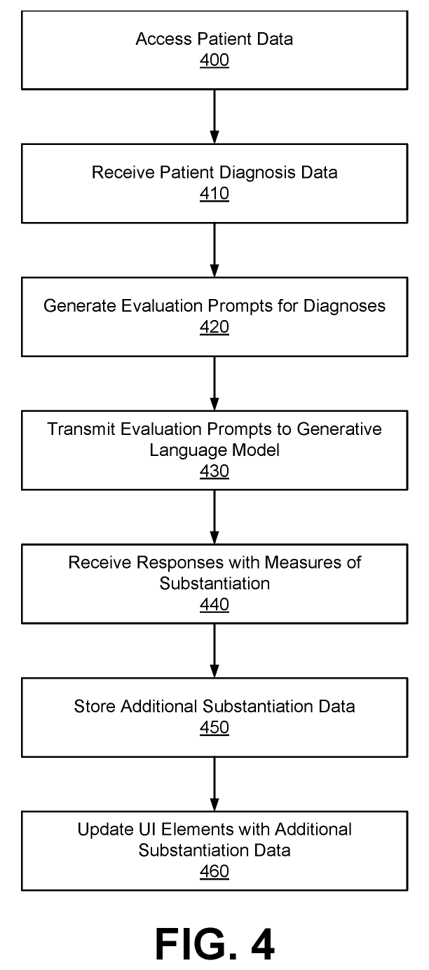

FIG. 4 is a flowchart for a method of generating additional substantiation data for patient diagnoses, in accordance with some embodiments. Alternative embodiments may include more, fewer, or different steps from those illustrated in FIG. 4, and the steps may be performed in a different order from that illustrated in FIG. 4. These steps may be performed by a physician documentation system (e.g., physician documentation system 120). Additionally, each of these steps may be performed automatically by the physician documentation system without human intervention.

The physician documentation system accesses 400 patient data for the patient. The patient data may include test results, such as laboratory test results or imaging results, as well as physician notes generated by a physician for prior appointments with the patient. The patient data may further include medication histories, referral records, and prior diagnosis records associated with the patient. The patient data may include a transcript of a session between the patient and a physician. The physician client device may record audio data of a session between the patient and the physician and may generate text data of the transcript from that audio data. For example, the physician client device may generate the transcript locally by applying a speech-to-text model to the audio data, or may transmit the audio data to the physician documentation system for transcription. The transcript may be a direct transcript of the session or may be a summarized version generated by applying a generative language model to the direct transcript.

The physician documentation system receives 410 patient diagnosis data for the patient through the user interface operating on the physician client device. The patient diagnosis data may include a list of diagnoses of the patient, such as International Classification of Diseases, Tenth Revision (ICD-10) codes or Hierarchical Condition Category (HCC) codes selected by the physician. The patient diagnosis data also includes initial substantiation data for each of the diagnoses. Initial substantiation data is substantiation data that is initially generated by the physician for a diagnosis. For example, the initial substantiation data for a diagnosis of hypertension may include physician notes describing the patient's elevated blood pressure readings recorded during the session, together with any prior health records that the physician has identified as relevant to that diagnosis. The physician documentation system may receive the patient diagnosis data from the physician client device and store the patient diagnosis data in association with the patient's health records.

The physician documentation system generates 420 a substantiation prompt for each of the diagnoses in the patient diagnosis data. A substantiation prompt is a prompt to a generative language model that instructs the generative language model to generate additional substantiation data for a diagnosis based on patient data. For example, the physician documentation system may generate a substantiation prompt that instructs the generative language model to generate additional text to supplement the physician notes describing the basis for a diagnosis, or to identify portions of the patient data to include as substantiation data for the diagnosis. Each substantiation prompt may include the corresponding diagnosis, the initial substantiation data for that diagnosis, the patient data accessed for the patient, and instructions to generate additional substantiation data for the diagnosis based on the initial substantiation data and the patient data. The substantiation prompt may also include diagnosis substantiation criteria for the corresponding diagnosis. For example, the substantiation prompt may include the Monitor, Evaluate, Assess, and Treat (MEAT) criteria, or diagnosis-specific clinical guidelines such as those published by third-party medical organizations, as described above. The physician documentation system may retrieve the diagnosis substantiation criteria for a diagnosis from a database and include those criteria in the substantiation prompt for that diagnosis.

The physician documentation system transmits 430 each of the substantiation prompts to a generative language model. The physician documentation system may transmit the substantiation prompts to the model serving system via the network. The physician documentation system may transmit the substantiation prompts sequentially, one for each diagnosis, or may transmit multiple substantiation prompts in parallel. The physician documentation system receives 440 a response from the generative language model for each of the substantiation prompts. Each response includes additional substantiation data for the corresponding diagnosis. The additional substantiation data may take the form of text to include in the physician notes for the diagnosis. The additional substantiation data may also take the form of an identification of one or more portions of the patient data to include as substantiation data for the diagnosis. The physician documentation system may extract the additional substantiation data from each response by parsing the text output generated by the generative language model.

In some embodiments, the physician documentation system may generate substantiation prompts selectively based on measures of sufficiency for the diagnoses. For example, the physician documentation system may generate a substantiation prompt only for a diagnosis whose measure of sufficiency falls below a threshold measure of sufficiency. The physician documentation system may compute the measure of sufficiency for each diagnosis, for example by performing the evaluation process described above with respect to FIGS. 2 or 3, and may generate substantiation prompts only for those diagnoses for which the measure of sufficiency does not meet the threshold measure of sufficiency. In some embodiments, the physician documentation system may also include the measure of sufficiency in the substantiation prompt for a diagnosis. The physician documentation system may include instructions in the substantiation prompt that direct the generative language model to use the measure of sufficiency when generating the additional substantiation data, so that the generative language model may tailor the additional substantiation data to address the specific deficiencies identified by the measure of sufficiency.

The physician documentation system stores 450 the additional substantiation data for the patient. The physician documentation system may store the additional substantiation data in association with the patient's health records and the corresponding diagnosis. The physician documentation system may store the additional substantiation data alongside the initial substantiation data for each diagnosis, so that both the initial substantiation data and the additional substantiation data are available for subsequent evaluations. The physician documentation system updates 460 the user interface element for each of the diagnoses in the user interface to include the additional substantiation data. For example, the physician documentation system may update a text box for physician notes to include the additional substantiation data generated by the generative language model, such that the text box displays both the initial substantiation data and the additional substantiation data together. The physician documentation system may also update the user interface element to highlight portions of the patient data that were identified by the generative language model as additional substantiation data for a diagnosis, so that the physician may readily identify which portions of the patient data have been added as substantiation data. In some embodiments, the physician documentation system may also update the user interface element to display a visual indicator distinguishing the additional substantiation data from the initial substantiation data, such as displaying the additional substantiation data in a different color or with an annotation indicating that the additional substantiation data was generated by the physician documentation system. The physician documentation system may transmit instructions to the physician client device to render the updated user interface elements, and the physician client device may update the display of the physician client application accordingly. In some embodiments, the user interface may also prompt the physician to review and verify the additional substantiation data before the additional substantiation data is saved to the patient's record.

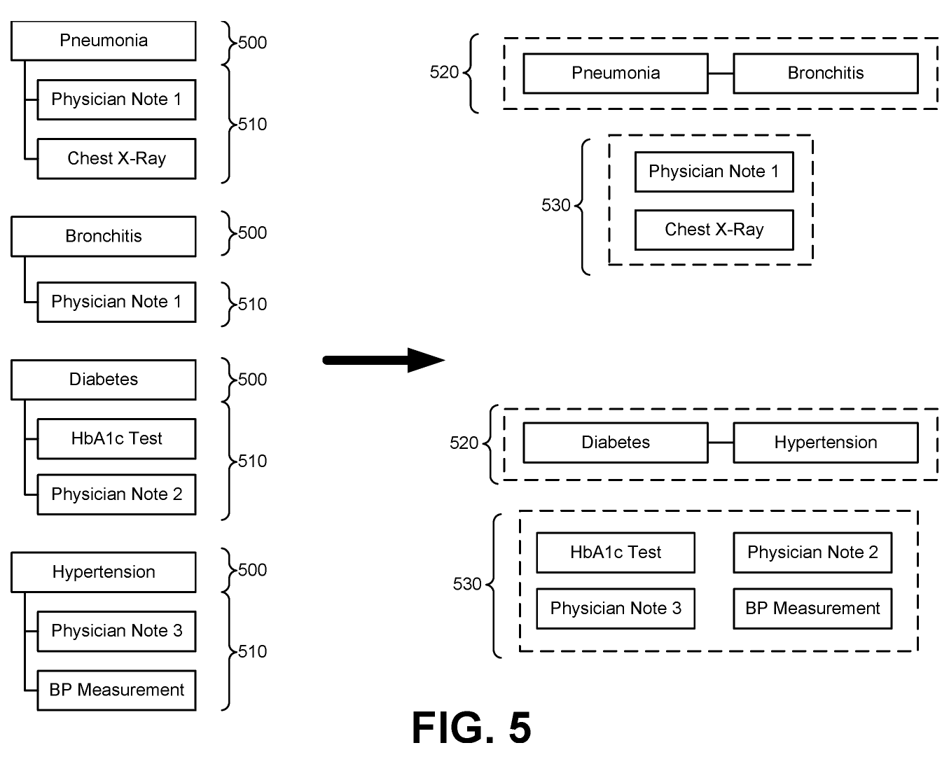

FIG. 5 illustrates an example of how the physician documentation system generates diagnosis clusters 520 from a set of diagnoses 500 and aggregates substantiation data 510 into substantiation data sets 530 for each of those diagnosis clusters 520, in accordance with some embodiments.

The left side of FIG. 5 illustrates a set of four diagnoses 500, each with associated substantiation data 510. For example, the diagnosis of Pneumonia has associated substantiation data 510 comprising Physician Note 1 and a Chest X-Ray, the diagnosis of Bronchitis has associated substantiation data 510 comprising Physician Note 1, the diagnosis of Diabetes has associated substantiation data 510 comprising an HbA1c Test and Physician Note 2, and the diagnosis of Hypertension has associated substantiation data 510 comprising Physician Note 3 and a BP Measurement.

The right side of FIG. 5 illustrates the result of the clustering process. The physician documentation system has generated two diagnosis clusters 520. The first diagnosis cluster 520 groups Pneumonia and Bronchitis together, reflecting a likelihood of comorbidity between those two diagnoses that exceeds the threshold likelihood of comorbidity. The second diagnosis cluster 520 groups Diabetes and Hypertension together, similarly reflecting a sufficiently high likelihood of comorbidity between those diagnoses. For each diagnosis cluster 520, the physician documentation system aggregates the substantiation data 510 associated with the diagnoses in that cluster into a substantiation data set 530. For example, the substantiation data set 530 for the Pneumonia-Bronchitis cluster includes Physician Note 1 and the Chest X-Ray, where Physician Note 1 appears only once in the substantiation data set 530 despite being associated with both Pneumonia and Bronchitis individually, illustrating the deduplication process described above. The substantiation data set 530 for the Diabetes-Hypertension cluster includes the HbA1c Test, Physician Note 2, Physician Note 3, and the BP Measurement, aggregating the substantiation data 510 from both diagnoses into a single substantiation data set 530 for evaluation.

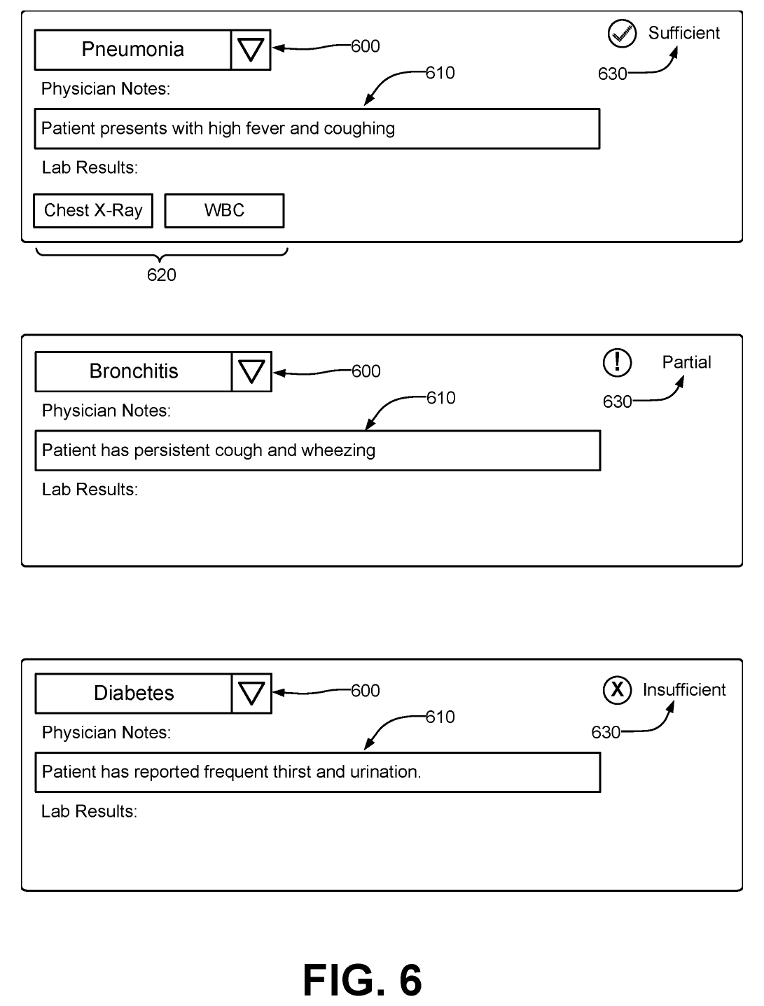

FIG. 6 illustrates example diagnosis user interface elements 600 displayed in the user interface of the physician client application, in accordance with some embodiments. FIG. 6 shows three diagnosis user interface elements 600, one for each of three diagnoses: Pneumonia, Bronchitis, and Diabetes. Each diagnosis user interface element 600 includes a physician notes user interface element 610 that displays the physician notes associated with the corresponding diagnosis, as well as a substantiation data user interface element 620 that displays the substantiation data associated with that diagnosis, such as laboratory test results or imaging results. Each diagnosis user interface element 600 also includes a sufficiency category user interface element 630 that displays the sufficiency category identified by the physician documentation system for the corresponding diagnosis.

FIG. 6 illustrates three different sufficiency categories that the physician documentation system may identify for a diagnosis. The diagnosis user interface element 600 for Pneumonia displays a sufficiency category user interface element 630 indicating "Sufficient," reflecting that the physician notes user interface element 610 for Pneumonia includes notes stating that the patient presents with high fever and coughing, and the substantiation data user interface element 620 includes a Chest X-Ray and a WBC test result. The diagnosis user interface element 600 for Bronchitis displays a sufficiency category user interface element 630 indicating "Partial," reflecting that the physician notes user interface element 610 includes notes stating that the patient has a persistent cough and wheezing, but the substantiation data user interface element 620 does not include any laboratory test results or imaging results for that diagnosis. The diagnosis user interface element 600 for Diabetes displays a sufficiency category user interface element 630 indicating "Insufficient," reflecting that the physician notes user interface element 610 includes only a note stating that the patient has reported frequent thirst and urination, and the substantiation data user interface element 620 does not include any supporting laboratory test results. The physician documentation system may update each sufficiency category user interface element 630 to display a visual indicator corresponding to the identified sufficiency category, such as a checkmark icon for a "Sufficient" category, a warning icon for a "Partial" category, and an error icon for an "Insufficient" category, so that the physician may readily identify which diagnoses require additional substantiation data.

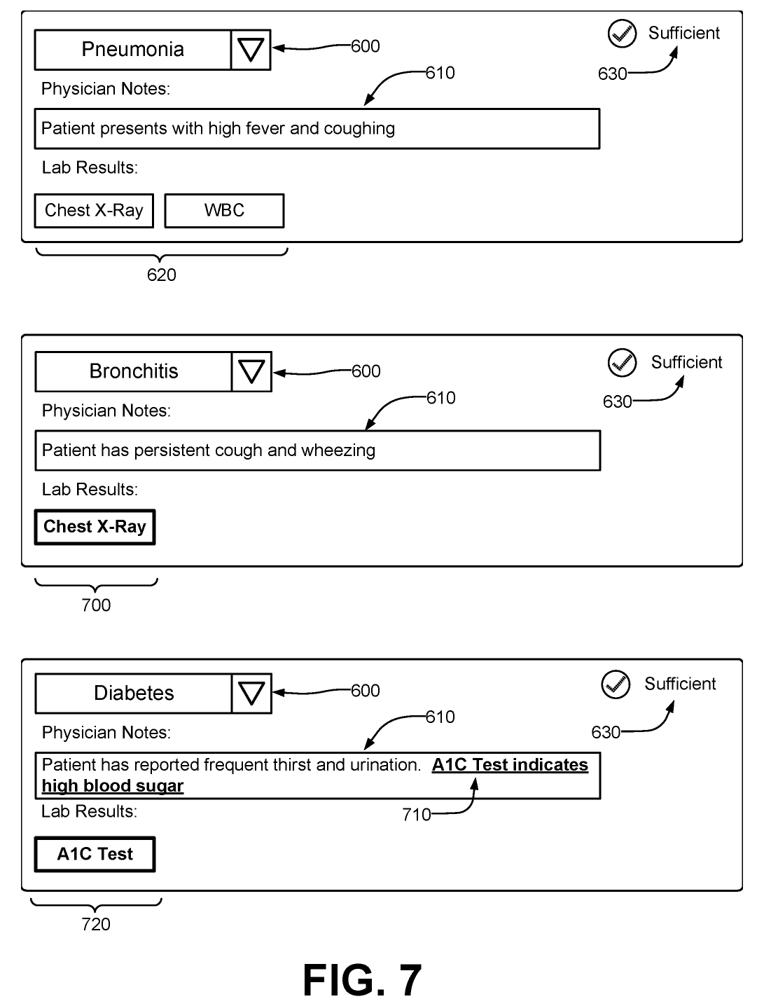

FIG. 7 illustrates additional substantiation data being displayed to a user through a user interface of a physician client application, in accordance with some embodiments. For the Bronchitis diagnosis, the physician documentation system has identified a Chest X-Ray as additional substantiation data 700 for that diagnosis and added the Chest X-Ray to the substantiation data for the Bronchitis diagnosis. For the Diabetes diagnosis, the physician documentation system has generated additional text 710 for the physician notes user interface element 610, including a note that an A1C Test indicates high blood sugar, supplementing the initial physician note stating that the patient has reported frequent thirst and urination. The physician documentation system has also identified an A1C Test as additional substantiation data 720 for the Diabetes diagnosis. The physician documentation system has updated the sufficiency category user interface elements 630 for Bronchitis and Diabetes to display "Sufficient," reflecting that the additional substantiation data generated by the generative language model is sufficient to substantiate those diagnoses.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor comprises one or more processors or processing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include a computer program product or other data combination described herein.

The description herein may describe processes and systems that use machine-learning models in the performance of their described functionalities. A “machine-learning model,” as used herein, comprises one or more machine-learning models that perform the described functionality. Machine-learning models may be stored on one or more computer-readable media with a set of weights. These weights are parameters used by the machine-learning model to transform input data received by the model into output data. The weights may be generated through a training process, whereby the machine-learning model is trained based on a set of training examples and labels associated with the training examples. The training process may include: applying the machine-learning model to a training example, comparing an output of the machine-learning model to the label associated with the training example, and updating weights associated with the machine-learning model through a back-propagation process. The weights may be stored on one or more computer-readable media, and are used by a system when applying the machine-learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or.” For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a non-limiting example, the condition “A, B, or C” is satisfied when A and B are true (or present) and C is false (or not present). Similarly, as another non-limiting example, the condition “A, B, or C” is satisfied when A is true (or present) and B and C are false (or not present).

1. A method comprising:
accessing, at an online system, patient data describing health characteristics of a patient;
receiving patient diagnosis data for the patient through a user interface operating on a physician client device, wherein the patient diagnosis data comprises a plurality of diagnoses of the patient and substantiation data for each of the plurality of diagnoses, wherein the substantiation data for a diagnosis of the plurality of diagnoses comprises a subset of the patient data that relates to the diagnosis;
generating a set of diagnosis clusters based on the plurality of diagnoses, wherein each of the set of diagnosis clusters comprises a subset of the plurality of diagnoses, and wherein generating a diagnosis cluster comprises identifying a subset of the plurality of diagnoses with likelihoods of comorbidity that exceed a threshold value;
aggregating the substantiation data into a substantiation data set for each of the set of diagnosis clusters by aggregating the substantiation data associated with the subset of diagnoses of each diagnosis cluster;
generating an evaluation prompt for each of the set of diagnosis clusters, wherein each of the evaluation prompts comprises the subset of diagnoses for a corresponding diagnosis cluster, a corresponding substantiation data set, and instructions to generate a measure of sufficiency of the substantiation data set for the subset of diagnoses based on the substantiation data;
transmitting each of the evaluation prompts to a generative language model;
receiving a response from the generative language model for each of the evaluation prompts, wherein each response comprises the measure of sufficiency for the corresponding diagnosis cluster; and
updating a user interface element for each of the plurality of diagnoses in the user interface based on the measures of sufficiency.

2. The method of claim 1, wherein the patient data comprises a transcript of a session between the patient and a physician.

3. The method of claim 1, wherein the substantiation data for each of the plurality of diagnoses comprises physician notes generated by the physician describing a basis for the corresponding diagnosis.

4. The method of claim 1, wherein each of the evaluation prompts further comprises diagnosis substantiation criteria for the diagnoses in the corresponding diagnosis cluster.

5. The method of claim 1, wherein generating a diagnosis cluster comprises:
computing likelihoods of comorbidity for pairs of diagnoses of the plurality of diagnoses; and
generating the diagnosis cluster based on the computed likelihoods of comorbidity.

6. The method of claim 5, wherein computing a likelihood of comorbidity for a pair of the plurality of diagnoses comprises:
computing a frequency with which the pair of diagnoses appear together based on patient data for a plurality of patients.

7. The method of claim 1, wherein generating a diagnosis cluster comprises:
identifying a subset of the plurality of diagnoses for which all pairwise likelihoods of comorbidity exceed the threshold value.

8. The method of claim 1, wherein generating a diagnosis cluster comprises:
adding a diagnosis to a diagnosis cluster based on a likelihood of comorbidity between the diagnosis and another diagnosis in the diagnosis cluster exceeds the threshold value.

9. The method of claim 1, wherein the measure of sufficiency is a numerical value representing a sufficiency of the substantiation data set for the corresponding diagnosis cluster.

10. The method of claim 1, wherein each of the evaluation prompts comprises instructions to generate a measure of sufficiency for each diagnosis in the corresponding diagnosis cluster based on the substantiation data set.

11. A non-transitory computer-readable medium storing computer-executable instructions that, when executed, cause a computing system to perform operations comprising:
accessing, at an online system, patient data describing health characteristics of a patient;
receiving patient diagnosis data for the patient through a user interface operating on a physician client device, wherein the patient diagnosis data comprises a plurality of diagnoses of the patient and substantiation data for each of the plurality of diagnoses, wherein the substantiation data for a diagnosis of the plurality of diagnoses comprises a subset of the patient data that relates to the diagnosis;
generating a set of diagnosis clusters based on the plurality of diagnoses, wherein each of the set of diagnosis clusters comprises a subset of the plurality of diagnoses, and wherein generating a diagnosis cluster comprises identifying a subset of the plurality of diagnoses with likelihoods of comorbidity that exceed a threshold value;
aggregating the substantiation data into a substantiation data set for each of the set of diagnosis clusters by aggregating the substantiation data associated with the subset of diagnoses of each diagnosis cluster;
generating an evaluation prompt for each of the set of diagnosis clusters, wherein each of the evaluation prompts comprises the subset of diagnoses for a corresponding diagnosis cluster, a corresponding substantiation data set, and instructions to generate a measure of sufficiency of the substantiation data set for the subset of diagnoses based on the substantiation data;
transmitting each of the evaluation prompts to a generative language model;
receiving a response from the generative language model for each of the evaluation prompts, wherein each response comprises the measure of sufficiency for the corresponding diagnosis cluster; and
updating a user interface element for each of the plurality of diagnoses in the user interface based on the measures of sufficiency.

12. The non-transitory computer-readable medium of claim 11, wherein the patient data comprises a transcript of a session between the patient and a physician.

13. The non-transitory computer-readable medium of claim 11, wherein the substantiation data for each of the plurality of diagnoses comprises physician notes generated by the physician describing a basis for the corresponding diagnosis.

14. The non-transitory computer-readable medium of claim 11, wherein each of the evaluation prompts further comprises diagnosis substantiation criteria for the diagnoses in the corresponding diagnosis cluster.

15. The non-transitory computer-readable medium of claim 11, wherein generating a diagnosis cluster comprises:
computing likelihoods of comorbidity for pairs of diagnoses of the plurality of diagnoses; and
generating the diagnosis cluster based on the computed likelihoods of comorbidity.

16. The non-transitory computer-readable medium of claim 15, wherein computing a likelihood of comorbidity for a pair of the plurality of diagnoses comprises:
computing a frequency with which the pair of diagnoses appear together based on patient data for a plurality of patients.

17. The non-transitory computer-readable medium of claim 11, wherein generating a diagnosis cluster comprises:
identifying a subset of the plurality of diagnoses for which all pairwise likelihoods of comorbidity exceed the threshold value.

18. The non-transitory computer-readable medium of claim 11, wherein generating a diagnosis cluster comprises:
adding a diagnosis to a diagnosis cluster based on a likelihood of comorbidity between the diagnosis and another diagnosis in the diagnosis cluster exceeds the threshold value.

19. The non-transitory computer-readable medium of claim 11, wherein the measure of sufficiency is a numerical value representing a sufficiency of the substantiation data set for the corresponding diagnosis cluster.

20. The non-transitory computer-readable medium of claim 11, wherein each of the evaluation prompts comprises instructions to generate a measure of sufficiency for each diagnosis in the corresponding diagnosis cluster based on the substantiation data set.
